SEQUENCE LISTING (21 sequences)

SEQ ID NO: 1 - protein, 117 aa, Artificial Sequence
  VH domain of antibody VEGF-0089
EVQLVESGGGLVQPGGSLRLSCAASGFTFTNYAMTWVRQAPGKGLEWVSSIGNGGGIYTYYADSVKGRFTISRDNSKNTLYLQMNSLRAEDTAVYYCAKGDNLFDSWGPGTLVTVSS

SEQ ID NO: 2 - protein, 106 aa, Artificial Sequence
  VL domain of antibodies VEGF-0089, VEGF-0112, VEGF-P1AE8674
DIQMTQSPASLSASVGDRVTITCRASQSIYSSLNWYQQKPGKAPKLLIYASTLQSGVPSRFSGSASGTDFTLTISSLQPEDVATYYCQQYQNFPRTFGQGTKLEIK

SEQ ID NO: 3 - protein, 5 aa, Artificial Sequence
  H-CDR1 of antibodies VEGF-0089, VEGF-0112, VEGF-P1AE8674
NYAMT

SEQ ID NO: 4 - protein, 18 aa, Artificial Sequence
  H-CDR2 of antibodies VEGF-0089, VEGF-P1AD8674
SIGNGGGIYTYYADSVKG

SEQ ID NO: 5 - protein, 7 aa, Artificial Sequence
  H-CDR3 of antibodies VEGF-0089, VEGF-0112, VEGF-P1AE8674
GDNLFDS

SEQ ID NO: 6 - protein, 11 aa, Artificial Sequence
  L-CDR1 of antibodies VEGF-0089, VEGF-0112, VEGF-P1AE8674
RASQSIYSSLN

SEQ ID NO: 7 - protein, 11 aa, Artificial Sequence
  L-CDR2 of antibodies VEGF-0089, VEGF-0112, VEGF-P1AE8674
ASTLQSGVPSR

SEQ ID NO: 8 - protein, 4 aa, Artificial Sequence
  L-CDR3 of antibodies VEGF-0089, VEGF-0112, VEGF-P1AE8674
FPRT

SEQ ID NO: 9 - protein, 31 aa, Artificial Sequence
  H-FR3 of VEGF-0089, VEGF-0112
FTISRDNSKNTLYLQMNSLRAEDTAVYYCAK

SEQ ID NO: 10 - protein, 235 aa, Artificial Sequence
  heavy chain of VEGF-0089 Fab fragment
EVQLVESGGGLVQPGGSLRLSCAASGFTFTNYAMTWVRQAPGKGLEWVSSIGNGGGIYTYYADSVKGRFTISRDNSKNTLYLQMNSLRAEDTAVYYCAKGDNLFDSWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSEQKLISEEDLGAAEPEA

SEQ ID NO: 11 - protein, 213 aa, Artificial Sequence
  light chain of VEGF-0089 Fab fragment
DIQMTQSPASLSASVGDRVTITCRASQSIYSSLNWYQQKPGKAPKLLIYASTLQSGVPSRFSGSASGTDFTLTISSLQPEDVATYYCQQYQNFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 117 aa, Artificial Sequence
  VH domain of antibody VEGF-0112
EVQLVESGGGLVQPGGSLRLSCAASGFTFTNYAMTWVRQAPGKGLEWVSSIGSGGGIYTYYADSVKGRFTISRDNSKNTLYLQMNSLRAEDTAVYYCAKGDNLFDSWGPGTLVTVSS

SEQ ID NO: 13 - protein, 18 aa, Artificial Sequence
  H-CDR2 of antibody VEGF-0112
SIGSGGGIYTYYADSVKG

SEQ ID NO: 14 - protein, 235 aa, Artificial Sequence
  heavy chain of VEGF-0112 Fab fragment
EVQLVESGGGLVQPGGSLRLSCAASGFTFTNYAMTWVRQAPGKGLEWVSSIGSGGGIYTYYADSVKGRFTISRDNSKNTLYLQMNSLRAEDTAVYYCAKGDNLFDSWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSEQKLISEEDLGAAEPEA

SEQ ID NO: 15 - protein, 117 aa, Artificial Sequence
  VH domain of antibody VEGF-P1AD8674
EVQLVESGGGLVQPGGSLRLSCAASGFTFTNYAMTWVRQAPGKGLEWVSSIGNGGGIYTYYADSVKGRFTISRNNAENTLYLQMNSLRAEDTAVYYCAKGDNLFDSWGPGTLVTVSS

SEQ ID NO: 16 - protein, 31 aa, Artificial Sequence
  H-FR3 of antibody VEGF-P1AD8674
FTISRNNAENTLYLQMNSLRAEDTAVYYCAK

SEQ ID NO: 17 - protein, 220 aa, Artificial Sequence
  heavy chain of VEGF-P1AD8674 Fab fragment
EVQLVESGGGLVQPGGSLRLSCAASGFTFTNYAMTWVRQAPGKGLEWVSSIGNGGGIYTYYADSVKGRFTISRNNAENTLYLQMNSLRAEDTAVYYCAKGDNLFDSWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 19 - protein, 214 aa, Artificial Sequence
  heavy chain of Lucentis (ranibizumab)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 226 aa, Artificial Sequence
  light chain of Lucentis (ranibizumab)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 121 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

(19) United States
(12) Patent Application Publication
(10) Pub. No.: US 2022/0002397 A1
(43) Pub. Date: Jan. 6, 2022

(54) METHOD FOR IMPROVING INHIBITION OF VEGF-BINDING TO VEGF-R1 OF AN ANTI-VEGF ANTIBODY

(71) Applicant: Hoffmann-La Roche Inc., Little Falls, NJ (US)

(72) Inventors: Joerg Benz, Basel (CH); Stefan Dengl, Penzberg (DE); Andreas Ehler, Basel (CH); Sebastian Fenn, Penzberg (DE); Joerg Moelleken, Penzberg (DE)

(73) Assignee: Hoffmann-La Roche Inc., Little Falls, NJ (US)

(21) Appl. No.: 17/350,273

(22) Filed: Jun. 17, 2021

Related U.S. Application Data
(63) Continuation of application No. PCT/EP2019/086510, filed on Dec. 20, 2019.

(30) Foreign Application Priority Data
Dec. 21, 2018 (EP) 18215020.1

(51) Int. Cl. C07K 16/22

(52) U.S. Cl. CPC C07K 16/22; C07K 2317/622; C07K 2317/51; C07K 2317/76; C07K 2317/55; C07K 2317/565; C07K 2317/567; C07K 2317/24

(57) ABSTRACT

The present invention relates to methods for modulating anti-VEGF antibodies in order to provide variants of an anti-VEGF antibody that exhibit improved inhibition of VEGF binding to VEGF-R1; and antibodies provided by said methods.

### CROSS REFERENCE TO RELATED APPLICATIONS

This application is a Continuation of International Application No. PCT/EP2019/086510, filed Dec. 20, 2019, claiming priority to European Application No. 18215020.1, filed Dec. 21, 2018, which are incorporated herein by reference in their entirety.

### SEQUENCE LISTING

This application contains a Sequence Listing which has been submitted electronically in ASCII format and is hereby incorporated by reference in its entirety. Said ASCII copy, created on May 27, 2021, is named Sequence_Listing.txt and is 22,616 bytes in size.

### FIELD OF THE INVENTION

The present invention relates to methods for modulating anti-VEGF antibodies in order to provide variants of an anti-VEGF antibody that exhibit improved inhibition of VEGF binding to VEGF-R1; and antibodies provided by said methods.

### BACKGROUND OF THE INVENTION

Anti-VEGF antibodies that are approved for clinical application, such as Avastin® and Lucentis®, inhibit VEGF-binding to both receptors, VEGF-R1 (FLT-1, fms-like tyrosine kinase) and VEGF-R2 (KDR/FLK-1, fetal liver kinase). VEGF-R1 and VEGF-R2 are closely related receptor tyrosine kinases (RTK).

There is a need for methods for modulating anti-VEGF antibodies to exhibit desired characteristics, such as inihibiting of VEGF-binding to both receptors.

### SUMMARY OF THE INVENTION

The present invention relates to a method of improving inhibition of VEGF binding to VEGF-R1 of an antibody that binds to VEGF comprising an antigen binding site formed by cognate pair of a VH and a VL domain, wherein the antibody binds to an epitope of VEGF that overlaps with the VEGF-R1-binding region and the VEGF-R2-binding region in the VEGF molecule. The method of the invention comprises (a) providing an analysis of the tertiary structure of a complex of a VEGF-dimer bound by a first and a second antigen binding site of said antibody that binds to VEGF (VEGF-dimer-antibody-complex), (b) identifying at least one amino acid residue located in the VH domain or VL domain of said antibody, wherein said amino acid residue within the first antigen binding site and said amino acid residue within the second antigen binding site are spatially arranged in close proximity, in the VEGF-dimer-antigen-complex; and (c) substituting said at least one amino acid residue identified in step b) by i) an amino acid having a smaller side chain volume; and/or ii) an amino acid having a side chain of different charge.

With the method of the invention inhibition of VEGF binding to VEGF-R1 mediated by certain anti-VEGF antibodies may be improved by a few modifications in their amino acid sequence, particularly in regions that are not involved in antigen binding.

In one embodiment the antibody binds to binds to the same or an overlapping epitope to a conformational epitope on a dimer of VEGF-A121, wherein VEGF-A121 comprises an amino acid sequence of SEQ ID NO: 21, wherein the epitope comprises in one of the individual VEGF-A121 molecules within the VEGF dimer amino acids F17, M18, D19, Y21, Q22, R23, Y25, H27, P28, I29, E30, M55, N62, L66, N100, K101, C102, E103, C104, R105 and P106; and in the other one of the individual VEGF-A121 molecules within the VEGF dimer amino acids E30, K48, M81 and Q87. In one embodiment the epitope is measured by x-ray crystallography.

In one embodiment the amino acid with a smaller side chain volume comprises a lower number of carbon atoms in the side chain than the amino acid that is substituted in step c).

In one embodiment the amino acid with a smaller side chain volume is selected from D, E, S, T, N, G, A, V, I, and L.

In one embodiment an amino acid with a side chain of positive charge is substituted by an amino acid having a side chain of negative charge or by an amino acid having an uncharged side chain.

In one embodiment an amino acid with a side chain of negative charge is substituted by an amino acid having a side chain of positive charge or by an amino acid having an uncharged side chain.

In one embodiment an amino acid with an uncharged side chain is substituted by an amino acid having a side chain of positive charge or by an amino acid having a side chain of negative charge.

In one embodiment the at least one substituted amino acid residue is located in the heavy chain variable domain of said antibody.

Another aspect of the invention is an antibody that binds to VEGF provided by a method of one of the invention.

Another aspect of the invention is an antibody that binds to VEGF and inhibits VEGF binding to VEGF-R1, provided by a method of the invention.

Another aspect of the invention an antibody that binds to VEGF having a VH domain comprising SEQ ID NO:12 and a VL domain comprising SEQ ID NO: 2.

Another aspect of the invention an antibody that binds to VEGF having a VH domain comprising SEQ ID NO:15 and a VL domain comprising SEQ ID NO: 2.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1: Inhibition of VEGF-binding to VEGF-R1 and VEGF-R2 in presence of antibody Fab fragments (VEGF:VEGF-R2/R1 inhibitition SPR) as described in Example 1.

FIG. 2: Crystal structure of VEGF dimer (purple) in complex with anti-VEGF antibody VEGF-0089 as determined by X ray crystallography according to Example 2. Red circle highlights regions in the VH domain of VEGF-0089 that are in close proximity.

FIG. 3: Epitope amino acids bound by VEGF-0089 Fab fragment in a dimer of VEGF-A121 (SEQ ID NO: 21) as determined by X ray crystallography according to Example 2. Amino acid positions comprised in each one of the VEGF-A121 molecules in contact with VEGF-0089 Fab fragment within a distance of 5 Å are highlighted in black.

FIG. 4: Overlay of the crystal structures of a human VEGF-A121-dimer in complex with VEGF-R1 domain 2 and a human VEGF-A121-dimer in complex with VEGF-0089 Fab as measured in Example 2.

FIG. 5: Overlay of the crystal structures of a human VEGF-A121-dimer in complex with VEGF-R2 domains 2 and 3 and a human VEGF-A121-dimer in complex with VEGF-0089 Fab as measured in Example 2.

FIG. 6: Inhibition of VEGF binding to VEGF-R1 in presence of anti-VEGF antibodies as described in Example 3 (0.34 nM VEGF).

FIG. 7: Inhibition of VEGF binding to VEGF-R1 in presence of anti-VEGF antibodies as described in Example 3 (0.7 nM VEGF).

FIG. 8: Inhibition of VEGF binding to VEGF-R2 in presence of anti-VEGF antibodies as described in Example 3 (0.34 nM VEGF).

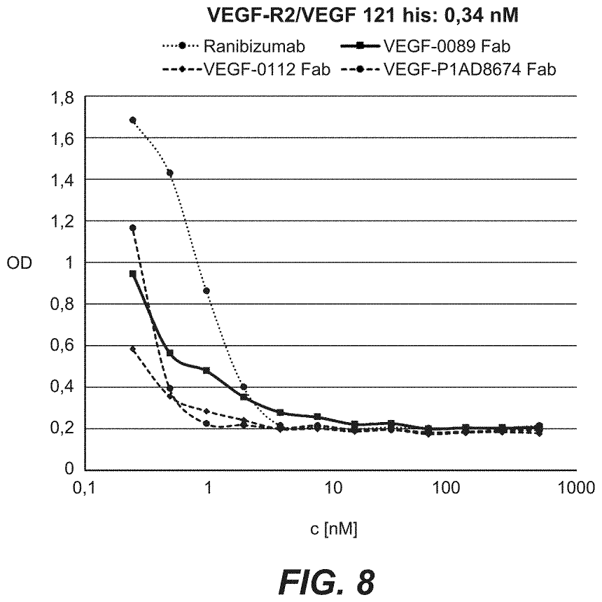

FIG. 9: Inhibition of VEGF binding to VEGF-R2 in presence of anti-VEGF antibodies as described in Example 3 (0.7 nM VEGF).

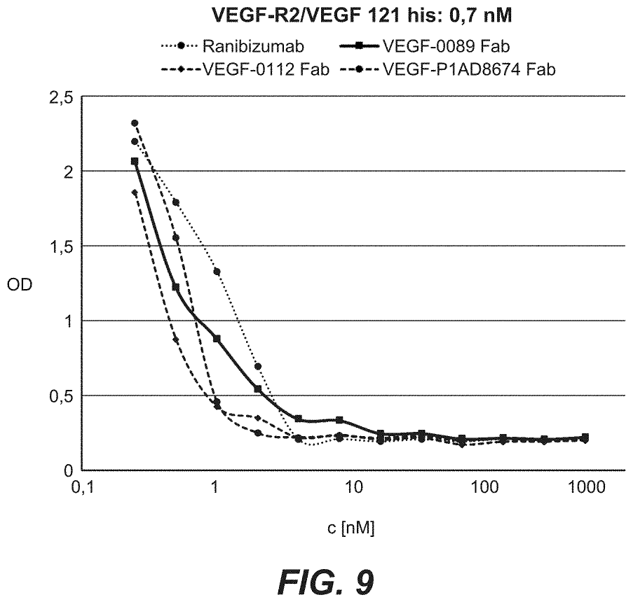

### DETAILED DESCRIPTION OF THE INVENTION

### 1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

Unless otherwise defined herein the term “comprising of” shall include the term “consisting of”.

The term “antibody” herein is used in the broadest sense and encompasses various antibody structures, including but not limited to monoclonal antibodies, multispecific antibodies (e.g., bispecific antibodies), and antibody fragments so long as they exhibit the desired antigen-binding activity.

Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs), including the complementarity determining regions (CDRs) (see, e.g., Kindt et al. Kuby Immunology, 6th ed., W.H. Freeman and Co., page 91 (2007)).

A “paratope” or an “antigen binding site”, as used interchangeably herein, refers to a part of an antibody which recognizes and binds to an antigen. An antigen binding site is formed by several individual amino acid residues from the antibody's heavy and light chain variable domains arranged that are arranged in spatial proximity in the tertiary structure of the Fv region. In one embodiment, the antigen binding site is defined as a set of the six CDRs comprised in a cognate VH/VL pair.

The term “complementarity determining regions” or “CDRs” as used herein refers to each of the regions of an antibody variable domain which are hypervariable in sequence and contain antigen-contacting residues. Generally, antibodies comprise six CDRs: three in the VH domain (CDR-H1, CDR-H2, CDR-H3), and three in the VL domain (CDR-L1, CDR-L2, CDR-L3). Unless otherwise indicated, CDR residues and other residues in the variable domain (e.g., FR residues) are numbered herein according to the Kabat numbering system (Kabat et al., Sequences of Proteins of Immunological Interest, 5th Ed. Public Health Service, National Institutes of Health, Bethesda, Md., 1991).

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below.

“Framework” or “FR” as used herein refers to variable domain amino acid residues other than CDR residues. The framework of a variable domain generally consists of four framework domains: FR1, FR2, FR3, and FR4. Accordingly, the CDR and FR amino acid sequences generally appear in the following sequence in the (a) VH domain: FR1-CDR-H1-FR2-CDR-H2-FR3-CDR-H3-FR4; and (b) in the VL domain: FR1-CDR-L1-FR2-CDR-L2-FR3-CDR-L3-FR4.

Vascular endothelial growth factor (VEGF) is a homodimeric member of the cystine knot family of growth factors. The term “VEGF”, as used herein, refers to any native VEGF from any vertebrate source, including mammals such as primates (e.g. humans) and rodents (e.g., mice and rats), unless otherwise indicated. The term encompasses “full-length”, unprocessed VEGF as well as any form of VEGF that results from processing in the cell. The term also encompasses naturally occurring variants of VEGF, e.g., splice variants or allelic variants. The amino acid sequence of an exemplary human VEGF is shown in SEQ ID NO:18. The term “VEGF-dimer” as used to herein refers to a homodimer of two identical VEGF-molecules. A complex formed by two identical antibody molecules that are bound to a VEGF-dimer is herein referred to as “VEGF-dimer-antibody-complex”.

A “first and a second antigen binding site” comprised in a VEGF-dimer-antibody-complex refers to the antigen binding site that is comprised in the VH/VL pair of each one of the two antibodies comprised in the VEGF-dimer-antibody-complex. For example, while the antigen binding site of one of the two anti-VEGF antibodies in the VEGF-dimer-antibody-complex is the “first antigen binding site”, the antigen binding site of other one of the two anti-VEGF antibodies is automatically the “second antigen binding site”.

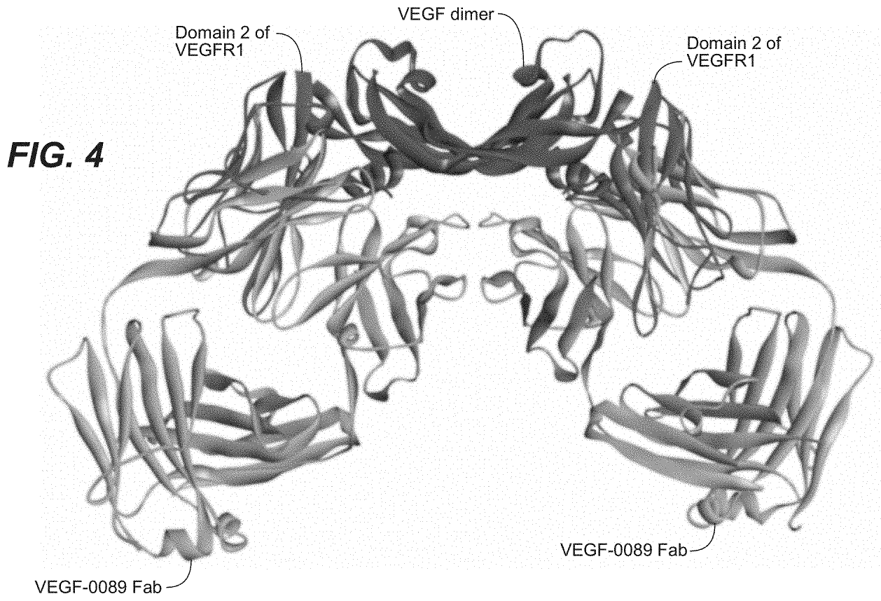

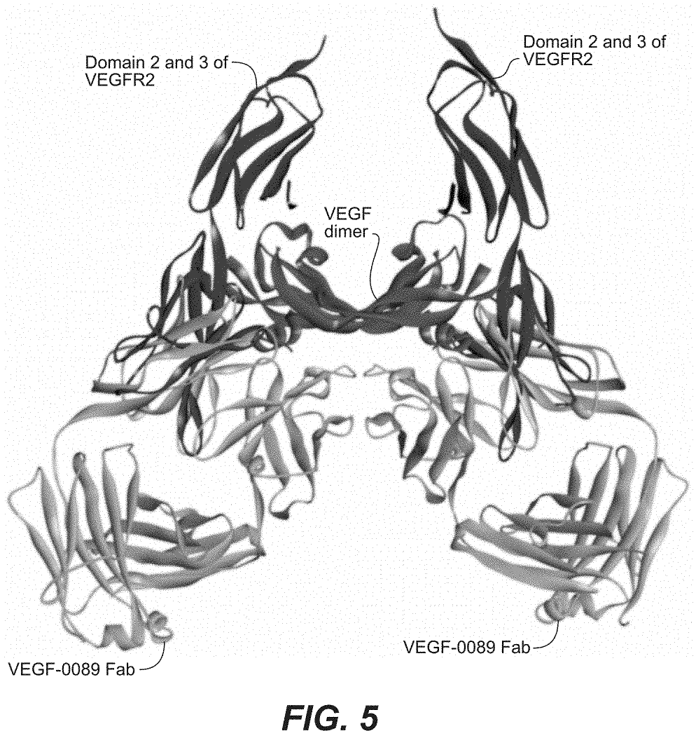

VEGF stimulates cellular responses by binding to tyrosine kinase receptors (the VEGF-receptors, or “VEGFRs”) on the cell surface, causing them to dimerize and become activated through transphosphorylation, although to different sites, times, and extents. VEGF-R1 and VEGF-R2 are closely related receptor tyrosine kinases (RTK). VEGF-A binds to VEGFR-1 (Flt-1), interacting with domain 2 of VEGF-R1, and VEGFR-2 (KDR/Flk-1), interacting with domains 2 and 3 of VEGF-R2 (see FIGS. 4 and 5).

The “VEGF-R1-binding region” and “VEGF-R2-binding region” of a VEGF molecule or a VEGF-dimer as used herein refers to those amino acids on the VEGF that interact with domain 2 of VEGF-R1 or domains 2 or 3 of VEGF-R2, respectively.

The terms “anti-VEGF antibody” and “an antibody that binds to VEGF” refer to an antibody that is capable of binding VEGF with sufficient affinity such that the antibody is useful as a diagnostic and/or therapeutic agent in targeting VEGF. In one embodiment, the extent of binding of an anti-VEGF antibody to an unrelated, non-VEGF protein is less than about 10% of the binding of the antibody to VEGF as measured, e.g., by surface plasmon resonance (SPR). In certain embodiments, an antibody that binds to VEGF has a dissociation constant (K_{D}) of ≤1 nM, or ≤0.15 nM. An antibody is said to “specifically bind” to VEGF when the antibody has a K_{D} of 1 μM or less.

Binding of an anti-VEGF antibody to VEGF may inhibit binding of VEGF to its receptors, VEGF-R1 or VEGF-R2, or both. Inhibition of binding of VEGF to VEGF-R1 or VEGF-R2 can be assessed by methods known in the art, such as ELISA or SPR.

The “tertiary structure” of a protein is the three dimensional shape of the protein. The tertiary structure exhibits a single polypeptide chain “backbone” with one or more protein secondary structures, the protein domains. Amino acid side chains may interact and bond in a number of ways. The interactions and bonds of side chains within a particular protein determine its tertiary structure. The “tertiary structure of a VEGF-dimer-antibody-complex” as used herein means the threedimensional shape of said complex.

Amino acid residues located “in close proximity” within a tertiary structure of a VEGF-dimer-antibody-complexes are amino acid residues derived from both anti-VEGF antibodies that are spatially arranged in the threedimensional shape of said complex in a way that their distance is up to 5 Å. This does not include amino acids adjacent to each other in the amino acid sequence of the individual domain, i.e. VH or VL, of the respective anti-VEGF antibody.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described herein.

The term “epitope” denotes the site on an antigen, either proteinaceous or non-proteinaceous, to which an anti-VEGF antibody binds. Epitopes can be formed both from contiguous amino acid stretches (linear epitope) or comprise non-contiguous amino acids (conformational epitope), e.g. coming in spatial proximity due to the folding of the antigen, i.e. by the tertiary folding of a proteinaceous antigen. Linear epitopes are typically still bound by an anti-VEGF antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., N.Y.).

Antigen Structure-based Antibody Profiling (ASAP), also known as Modification-Assisted Profiling (MAP), allows to bin a multitude of monoclonal antibodies specifically binding to VEGF based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Also competitive binding can be used to easily determine whether an antibody binds to the same epitope of VEGF as, or competes for binding with, a reference anti-VEGF antibody. For example, an “antibody that binds to the same epitope” as a reference anti-VEGF antibody refers to an antibody that blocks binding of the reference anti-VEGF antibody to its antigen in a competition assay by 50% or more, and conversely, the reference antibody blocks binding of the antibody to its antigen in a competition assay by 50% or more. Also for example, to determine if an antibody binds to the same epitope as a reference anti-VEGF antibody, the reference antibody is allowed to bind to VEGF under saturating conditions. After removal of the excess of the reference anti-VEGF antibody, the ability of an anti-VEGF antibody in question to bind to VEGF is assessed. If the anti-VEGF antibody is able to bind to VEGF after saturation binding of the reference anti-VEGF antibody, it can be concluded that the anti-VEGF antibody in question binds to a different epitope than the reference anti-VEGF antibody. But, if the anti-VEGF antibody in question is not able to bind to VEGF after saturation binding of the reference anti-VEGF antibody, then the anti-VEGF antibody in question may bind to the same epitope as the epitope bound by the reference anti-VEGF antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to VEGF, then it can be concluded that the anti-VEGF antibody in question and the reference anti-VEGF antibody compete for binding to VEGF.

In some embodiments two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some embodiments two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding” an antibody refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

Amino acids may be grouped according to common side-chain properties: (1) hydrophobic side chains: Norleucine, Met (M), Ala (A), Val (V), Leu (L), Ile (I); uncharged hydrophilic side chains (also referred to in the art as “neutral” hydrophilic side chains): Cys (C), Ser (S), Thr (T), Asn (N), Gln (Q); negatively charged side chains (also referred to in the art as “acidic” side chains): Asp (D), Glu (E); positively charged side chains (also referred to in the art as “basic” side chains): His (H), Lys (K), Arg (R); aromatic side chains: Trp (W), Tyr (Y), Phe (F); and side chains that comprise residues that influence chain orientation: Gly (G), Pro (P).

When referring to amino acid modifications, amino acids “having a smaller side chain volume” refer to amino acids that have a smaller side chain volume than the original amino acid located at the position to be modified. In certain embodiments, amino acids having a smaller side chain volume comprise a lower number of carbon atoms in the side chain than the original amino acid located at the position to be modified.

### 2. Detailed Description of the Embodiments of the Invention

The present invention relates to a method improving inhibition of VEGF binding to VEGF-R1 of an antibody that binds to VEGF comprising an antigen binding site formed by cognate pair of a VH and a VL domain, wherein the antibody binds to an epitope of VEGF that overlaps with the VEGF-R1-binding region and the VEGF-R2-binding region in the VEGF molecule. The method of the invention comprises (a) providing an analysis of the tertiary structure of a complex of a VEGF-dimer bound by a first and a second antigen binding site of said antibody that binds to VEGF (VEGF-dimer-antibody-complex), (b) identifying at least one amino acid residue located in the VH domain or VL domain of said antibody, wherein said amino acid residue within the first antigen binding site and said amino acid residue within the second antigen binding site are spatially arranged in close proximity in the VEGF-dimer-antigen-complex; and (c) substituting said at least one amino acid residue identified in step b) by i) an amino acid having a smaller side chain volume; and/or ii) an amino acid having a side chain of different charge.

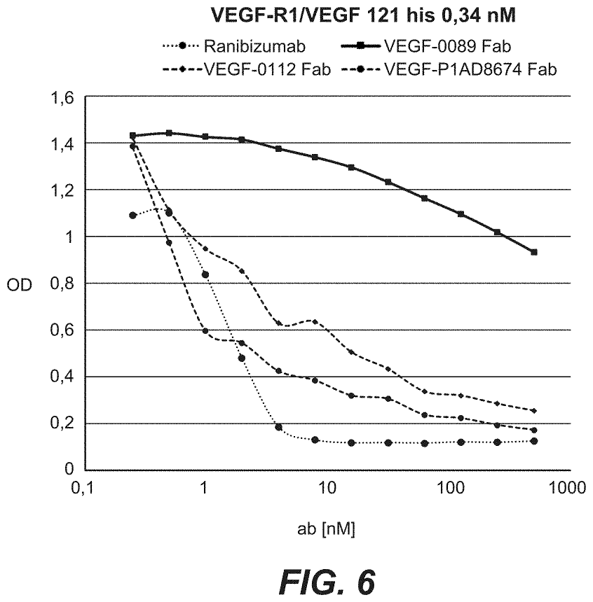

The method of the invention is for modulating anti-VEGF antibodies that bind to an epitope of VEGF that overlaps with the VEGF-R1-binding region and the VEGF-R2-binding region in the VEGF molecule. In one embodiment said epitope comprises amino acids interacting with domain 2 of VEGF-R1, when VEGF is bound to VEGF-R1. In one embodiment said epitope comprises amino acids interacting with domains 2 and 3 of VEGF-R2, when VEGF is bound to VEGF-R1. In one embodiment said anti-VEGF antibody binds to an epitope that overlaps with the epitope bound by an antibody characterized by a VH of SEQ ID NO:01 and a VL of SEQ ID NO:02 (antibody VEGF-0089 as described herein). In one embodiment said anti-VEGF antibody binds to the same epitope than antibody VEGF-0089 as described herein, as measured by x-ray crystallography. In one embodiment said anti-VEGF antibody binds to the same epitope than antibody VEGF-0089 as described herein, as measured by x-ray crystallography as described in Example 3. In one embodiment, said epitope is a conformational epitope within a dimer of VEGF-A121, wherein VEGF-A121 comprises an amino acid sequence of SEQ ID NO: 21, wherein the epitope comprises in one of the individual VEGF-A121 molecules within the VEGF dimer amino acids F17, M18, D19, Y21, Q22, R23, Y25, H27, P28, I29, E30, M55, N62, L66, N100, K101, C102, E103, C104, R105 and P106; and in the other one of the individual VEGF-A121 molecules within the VEGF dimer amino acids E30, K48, M81 and Q87. The numbering is according to the position of the amino acid in the amino acid sequence of VEGF-A121 indicated in SEQ ID NO: 21 (see also FIG. 6).

Within a method of the invention an analysis of the tertiary structure of a VEGF-dimer-antibody-complex is provided. The tertiary structure may be provided by methods known in the art. In one embodiment the tertiary structure is provided by x-ray crystallography, e.g. as described in Example 3 herein.

In a method of the invention amino acid residues in the VH domain and/or VL domain of said antibody are identified that are in close proximity within the VEGF-dimer-antibody-complex. In one embodiment “close proximity” refers to a distance of 10 Å or less. In one embodiment “close proximity” refers to a distance of 5 Å or less. This at least one amino acid residue is considered suitable for modification.

In one embodiment such at least one amino acid residue identified in step (b) is located in the heavy chain variable domain of said antibody. In one embodiment the at least one amino acid residue identified in step (b) is located within a heavy chain CDR of said antibody. In one embodiment the at least one amino acid residue identified in step (b) is located within H-CDR2 of said antibody. In one embodiment the at least one amino acid residue identified in step (b) is located within a heavy chain FR of said antibody. In one embodiment the at least one amino acid residue identified in step (b) is located within H-FR3 of said antibody.

In the method of the invention such amino acid residue is substituted by an amino acid having a smaller side chain volume than the amino acid residue comprised in the anti-VEGF antibody to be improved. Alternatively, such amino acid residue is substituted by an amino acid having a side chain of different charge. In certain embodiments, more than one amino acid residue is substituted in step (c) of the method of the invention. In this case, such at least two amino acid residues may be substituted independently from each other by an amino acid having a smaller side chain volume or by an amino acid having a side chain of different charge.

In one embodiment said amino acid having a smaller side chain volume is is selected from D, E, S, T, N, G, A, V, I, and L.

In one embodiment an amino acid is substituted with an amino acid having a side chain of different charge. In one embodiment an amino acid selected from R, K and H is substituted by an amino acid selected from D and E. In one embodiment an amino acid selected from R, K and H is substituted by an amino acid selected from C, S, T, N and Q. In one embodiment an amino acid selected from E and D is substituted by an amino acid selected from K, R and H. In one embodiment an amino acid selected from E and D is substituted by an amino acid selected from C, S, T, N and Q. In one embodiment an amino acid selected from C, S, T, N and Q is substituted by an amino acid selected from K, R and H. In one embodiment an amino acid selected from C, S, T, N and Q is substituted by an amino acid selected from E and D.

In one embodiment said anti-VEGF antibody of step a) binds to human VEGFA. In one embodiment said anti-VEGF antibody of step a) binds to human VEGF of SEQ ID NO:18.

In one aspect the anti-VEGF antibody improved by a method of the invention comprises (a) CDR-H1 comprising the amino acid sequence of SEQ ID NO:03; (b) CDR-H2 comprising the amino acid sequence of SEQ ID NO:04; (c) CDR-H3 comprising the amino acid sequence of SEQ ID NO:05; (d) CDR-L1 comprising the amino acid sequence of SEQ ID NO:06; (e) CDR-L2 comprising the amino acid sequence of SEQ ID NO:07; and (0 CDR-L3 comprising the amino acid sequence of SEQ ID NO:08. One exemplary antibody comprising this set of CDR amino acid sequences is the antibody referred to herein as “VEGF-0089”. In one embodiment said antibody is characterized by a VH comprising SEQ ID NO:01 and a VL comprising SEQ ID NO:02.

In one embodiment of this aspect, the at least one amino acid residue identified in step (b) is located within H-CDR2 of said antibody. In one embodiment said at least one amino acid residue is Kabat position 52a. In one embodiment an amino acid residue from Kabat position 52a is modified by substitutions with C, S, T, N or Q. In one embodiment step c) includes an N52aS substitution.

In one embodiment of this aspect, the at least one amino acid residue identified in step (b) is located within H-FR3 of said antibody. In one embodiment said at least one amino acid residue is selected from Kabat position 72, 74, and 75. In one embodiment one, two or three amino acids of Kabat position 72, 74, and 75 are modified. In one embodiment Kabat position 72 is modified by substitutions with K, R, H, C, S, T, N or Q. In one embodiment Kabat position 72 is modified by substitutions with N. In one embodiment Kabat position 74 is modified by substitutions with A. In one embodiment Kabat position 75 is modified by substitutions with D, E, C, S, T, N or Q. In one embodiment Kabat position 75 is modified by substitutions with D or E.

In one embodiment of the invention, following step (c) the method comprises a step (d) of comparing VEGF binding to VEGF-R1 of the anti-VEGF antibody comprising the substitutions introduced in step (c) and the anti-VEGF antibody not comprising said substitutions (i.e. the parental anti-VEGF antibody).

The present invention also relates to an anti-VEGF antibody provided by a method of the invention. The present invention further relates to an anti-VEGF antibody that binds to VEGF and inhibits VEGF binding to VEGF-R1, provided by a method of the invention. In one embodiment the antibody provided by a method of the invention is an isolated antibody.

Such anti-VEGF antibodies may be produced using recombinant methods known in the art, e.g., as described in U.S. Pat. No. 4,816,567, e.g. recombinant expression in eukaryotic cell such as HEK293 cells as described in Example 1. For these methods one or more isolated nucleic acid(s) encoding an antibody are provided.

In certain embodiments, an antibody provided herein is an antibody fragment. In one embodiment, the antibody fragment is a Fab, Fab′, Fab′-SH, or F(ab′)₂ fragment, in particular a Fab fragment.

In certain embodiments, an antibody provided herein is a full length antibody.

In one embodiment the antibody is an IgG1 antibody.

The present invention further relates to a nucleic acid encoding for an anti-VEGF antibody provided by a method of the invention. The present invention further relates to a host cell comprising the nucleic acid of the invention.

### DESCRIPTION OF THE AMINO ACID SEQUENCES

| | |
|---|---|
| SEQ ID NO: 1 | VH domain of antibody VEGF-0089 |
| | EVQLVESGGGLVQPGGSLRLSCAASGFTFTNYAMTWVRQAPG |
| | KGLEWVSSIGNGGGIYTYYADSVKGRFTISRDNSKNTLYLQM |
| | NSLRAEDTAVYYCAKGDNLFDSWGPGTLVTVSS |
| SEQ ID NO: 2 | VL domain of antibodies VEGF-0089, VEGF-0112, |
| | VEGF-P1AE8674 |
| | DIQMTQSPASLSASVGDRVTITCRASQSIYSSLNWYQQKPGK |
| | APKLLIYASTLQSGVPSRFSGSASGTDFTLTISSLQPEDVAT |
| | YYCQQYQNFPRTFGQGTKLEIK |
| SEQ ID NO: 3 | H-CDR1 of antibodies VEGF-0089, VEGF-0112, |
| | VEGF-P1AE8674 |
| | NYAMT |
| SEQ ID NO: 4 | H-CDR2 of antibodies VEGF-0089, VEGF-P1AD8674 |
| | SIGNGGGIYTYYADSVKG |
| SEQ ID NO: 5 | H-CDR3 of antibodies VEGF-0089, VEGF-0112, |
| | VEGF-P1AE8674 |
| | GDNLFDS |
| SEQ ID NO: 6 | L-CDR1 of antibodies VEGF-0089, VEGF-0112, |
| | VEGF-P1AE8674 |
| | RASQSIYSSLN |
| SEQ ID NO: 7 | L-CDR2 of antibodies VEGF-0089, VEGF-0112, |
| | VEGF-P1AE8674 |
| | ASTLQSGVPSR |
| SEQ ID NO: 8 | L-CDR3 of antibodies VEGF-0089, VEGF-0112, |
| | VEGF-P1AE8674 |
| | FPRT |
| SEQ ID NO: 9 | H-FR3 of VEGF-0089, VEGF-0112 |
| | FTISRDNSKNTLYLQMNSLRAEDTAVYYCAK |
| SEQ ID NO: 10 | heavy chain of VEGF-0089 Fab fragment |
| | EVQLVESGGGLVQPGGSLRLSCAASGFTFTNYAMTWVRQAPG |
| | KGLEWVSSIGNGGGIYTYYADSVKGRFTISRDNSKNTLYLQM |
| | NSLRAEDTAVYYCAKGDNLFDSWGPGILVTVSSGQPKAPSVF |
| | PLAPCCGDTPSSTVTLGCLVKGYLPEPVTVIWNSGTLINGVR |
| | TFPSVRQSSGLYSLSSVVSVISSSQPVICNVAHPATNTKVDK |
| | TVAPSTCSEQKLISEEDLGAAEPEA |
| SEQ ID NO: 11 | light chain of VEGF-0089 |
| | DIQMTQSPASLSASVGDRVTITCRASQSIYSSLNWYQQKPGK |
| | APKLLIYASTLQSGVPSRFSGSASGTDFTLTISSLQPEDVAT |
| | YYCQQYQNFPRTFGQGTKLEIKRTVAAPSVFIFPPSDEQLKS |
| | GTASVVCLLNNFYPREAKVQWKVDNALQSGNSQESVTEQDSK |
| | DSTYSLSSTLTLSKADYEKHKVYACEVTHQGLSSPVTKSFNR |
| | GEC |
| SEQ ID NO: 12 | VH domain of antibody VEGF-0112 |
| | EVQLVESGGGLVQPGGSLRLSCAASGFTFTNYAMTWVRQAPG |
| | KGLEWVSSIGSGGGIYTYYADSVKGRFTISRDNSKNTLYLQM |
| | NSLRAEDTAVYYCAKGDNLFDSWGPGTLVTVSS |
| SEQ ID NO: 13 | H-CDR2 of antibody VEGF-0112 |
| | SIGSGGGIYTYYADSVKG |
| SEQ ID NO: 14 | heavy chain of VEGF-0112 Fab fragment |
| | EVQLVESGGGLVQPGGSLRLSCAASGFTFTNYAMTWVRQAPG |
| | KGLEWVSSIGSGGGIYTYYADSVKGRFTISRDNSKNTLYLQM |
| | NSLRAEDTAVYYCAKGDNLFDSWGPGILVTVSSGQPKAPSVF |
| | PLAPCCGDTPSSTVTLGCLVKGYLPEPVTVTWNSGTLINGVR |
| | TFPSVRQSSGLYSLSSVVSVISSSQPVICNVAHPATNTKVDK |
| | TVAPSTCSEQKLISEEDLGAAEPEA |
| SEQ ID NO: 15 | VH domain of antibody VEGF-P1AD8674 |
| | EVQLVESGGGLVQPGGSLRLSCAASGFTFTNYAMTWVRQAPG |
| | KGLEWVSSIGNGGGIYTYYADSVKGRFTISRNNAENTLYLQM |
| | NSLRAEDTAVYYCAKGDNLFDSWGPGTLVTVSS |
| SEQ ID NO: 16 | H-FR3 of antibody VEGF-P1AD8674 |
| | FTISRNNAENTLYLQMNSLRAEDTAVYYCAK |
| SEQ ID NO: 17 | heavy chain of VEGF-P1AD8674 Fab fragment |
| | EVQLVESGGGLVQPGGSLRLSCAASGFTFTNYAMTWVRQAPG |
| | KGLEWVSSIGNGGGIYTYYADSVKGRFTISRNNAENTLYLQM |
| | NSLRAEDTAVYYCAKGDNLFDSWGPGILVTVSSASTKGPSVF |
| | PLAPSSKSTSGGTAALGCLVKDYFPEPVTVSWNSGALTSGVH |
| | TFPAVLQSSGLYSLSSVVTVPSSSLGTQTYICNVNHKPSNTK |
| | VDKKVEPKSC |
| SEQ ID NO: 18 | human VEGF |
| | MNFLLSWVHWSLALLLYLHHAKWSQAAPMAEGGGQNHHEVVK |
| | FMDVYQRSYCHPIETLVDIFQEYPDEIEYIFKPSCVPLMRCG |
| | GCCNDEGLECVPTEESNITMQIMRIKPHQGQHIGEMSFLQHN |
| | KCECRPKKDRARQEKKSVRGKGKGQKRKRKKSRYKSWSVYVG |
| | ARCCLMPWSLPGPHPCGPCSERRKHLFVQDPQTCKCSCKNTD |
| | SRCKARQLELNERTCRCDKPRR |
| SEQ ID NO: 19 | heavy chain of Lucentis (ranibizumab) |
| | DIQLTQSPSSLSASVGDRVTITCSASQDISNYLNWYQQKPGK |
| | APKVLIYFTSSLHSGVPSRFSGSGSGTDFTLTISSLQPEDFA |
| | TYYCQQYSTVPWTFGQGTKVEIKRTVAAPSVFIFPPSDEQLK |
| | SGTASVVCLLNNFYPREAKVQWKVDNALQSGNSQESVTEQDS |
| | KDSTYSLSSILTLSKADYEKHKVYACEVTHQGLSSPVTKSFN |
| | RGEC |
| SEQ ID NO: 20 | light chain of Lucentis (ranibizumab) |
| | EVQLVESGGGLVQPGGSLRLSCAASGYDFTHYGMNWVRQAPG |
| | KGLEWVGWINTYTGEPTYAADFKRRFTFSLDTSKSTAYLQMN |
| | SLRAEDTAVYYCAKYPYYYGTSHWYFDVWGQGTLVTVSSAST |
| | KGPSVFPLAPSSKSTSGGTAALGCLVKDYFPEPVTVSWNSGA |
| | LTSGVHTFPAVLQSSGLYSLSSVVTVPSSSLGTQTYICNVNH |
| | KPSNTKVDKKVEPKSC |
| SEQ ID NO: 21 | Human VEGF-A121 |
| | APMAEGGGQNHHEVVKFMDVYQRSYCHP1ETLVDIFQEYPDE |
| | IEYIFKPSCVPLMRCGGCCNDEGLECVPTEESNITMQIMRIK |
| | PHQGQHIGEMSFLQHNKCECRPKKDRARQEKCDKPRR |

### EXAMPLES

The following examples are provided to aid the understanding of the present invention, the true scope of which is set forth in the appended claims. It is understood that modifications can be made in the procedures set forth without departing from the spirit of the invention.

### Example 1

### Characterisation of Generated Human Anti-VEGF Antibody (Antibody VEGF-0089)

Antibody “VEGF-0089” as used herein was derived from Roche proprietary transgenic rabbits as outlined in EP 17211032.2. VEGF-0089 comprises a VH domain of SEQ ID NO:01 and a VL domain of SEQ ID NO:02. For the subsequent analyses the antibody VEGF-0089 was generated as a Fab fragment (herein referred to as “VEGF-0089 Fab fragment” or simply “VEGF-0089 Fab”) having the human VH and VL domains and rabbit derived constant domains of the light chain (CLkappa) and heavy chain (CH1). The amino acid sequence of the heavy chain of VEGF-0089 Fab fragment is SEQ ID NO:10. The amino acid sequence of the light chain of VEGF-0089 Fab fragment is SEQ ID NO:11.

For recombinant expression of the antibody PCR-products coding for VH or VL were cloned as cDNA into expression vectors and transiently transformed into HEK-293 cells.

VEGF-binding of antibody VEGF-0089 Fab fragment was assessed by surface plasmon resonance (SPR) as described below.

### Determination of Antibody Binding Affinity by Surface Plasmon Resonance (SPR)

An anti-His capturing antibody (GE Healthcare 28995056) was immobilized to a Series S Sensor Chip C1 (GE Healthcare 29104990) using standard amine coupling chemistry resulting in a surface densitiy of 500-1000 resonance units (RU). As running and dilution buffer, HBS-P+(10 mM HEPES, 150 mM NaCl pH 7.4, 0.05% Surfactant P20) was used, the measurement temperature was set to 25° C. and 37° C., respectively. hVEGF-A121 was captured to the surface with resulting capture levels ranging from 5 to 35 RU. Dilution series of anti-VEGF antibodies (0.37-30 nM) were injected for 120 s, dissociation was monitored for at least 600 s at a flow rate of 30 μl/min. The surface was regenerated by injecting 10 mM Glycine pH 1.5 for 60 s. Bulk refractive index differences were corrected by subtracting blank injections and by subtracting the response obtained from the control flow cell without captured hVEGF-A121. Rate constants were calculated using the Langmuir 1:1 binding model within the Biacore Evaluation software.

As a result, the K_{D} of the VEGF-0089 Fab fragment was determined to be 134 pM (at a temperature of 25° C.).

For further characterization of the antibody, inhibition of VEGF-binding to its receptors VEGF-R1 and VEGF-R2 in presence of VEGF-0089 Fab fragment was assessed as described below:

### Inhibition of VEGF-Binding to VEGF-R1 and VEGF-R2 in Presence of Antibody Fab Fragments (VEGF: VEGF-R2/R1 Inhibition ELISA)

384 well streptavidin plates (Nunc/Microcoat #11974998001) were coated with 0.25 μg/mlbiotinylated VEGF-R1 or 0.5 m/ml biotinylated VEGF-R2 (inhouse production, each 25 μl/well in DPBS (1×) (PAN, #PO4-36500)). Plates were incubated for 1 h at room temperature. In parallel, VEGF-121-His (inhouse production) at a concentration of 0.7 nM was incubated with antibodies in different dilutions (12×1:2 dilution steps, starting with a concentration of 500 nM). This pre-incubation step was carried out in 384 well PP plates (Weidmann medical technology, #23490-101) in 1×OSEP buffer (bidest water, 10×, Roche, #11 666 789 001+0.5% Bovine Serum Albumin Fraction V, fatty acid free, Roche, #10 735 086 001+0.05% Tween 20). Plates were incubated for 1 h at room temperature. After washing VEGF-R1/VEGF-R2 coated streptavidin plates 3 times with 90 μl/well PBST-buffer (bidest water, 10×PBS Roche #11666789001+0.1% Tween 20), 25 μl of samples from the VEGF-antibody pre-incubation plate were transferred to coated strepavidin plates which were subsequently incubated for 1 h at room temperature. After washing 3 times with 90 μl/well PBST-buffer, 25 μl/well detection antibody (anti His POD, Bethyl, #A190-114P, 1:12000) in 1×OSEP was added. After incubation for 1 h at room temperature plates were washed 3 times with 90 μl PBST-buffer. 25 μl TMB (Roche, #11 835 033 001) was added to all wells simultaneously. After 10 min incubation at room temperature, signals were detected at 370 nm/492 nm on a Tecan Safire 2 Reader.

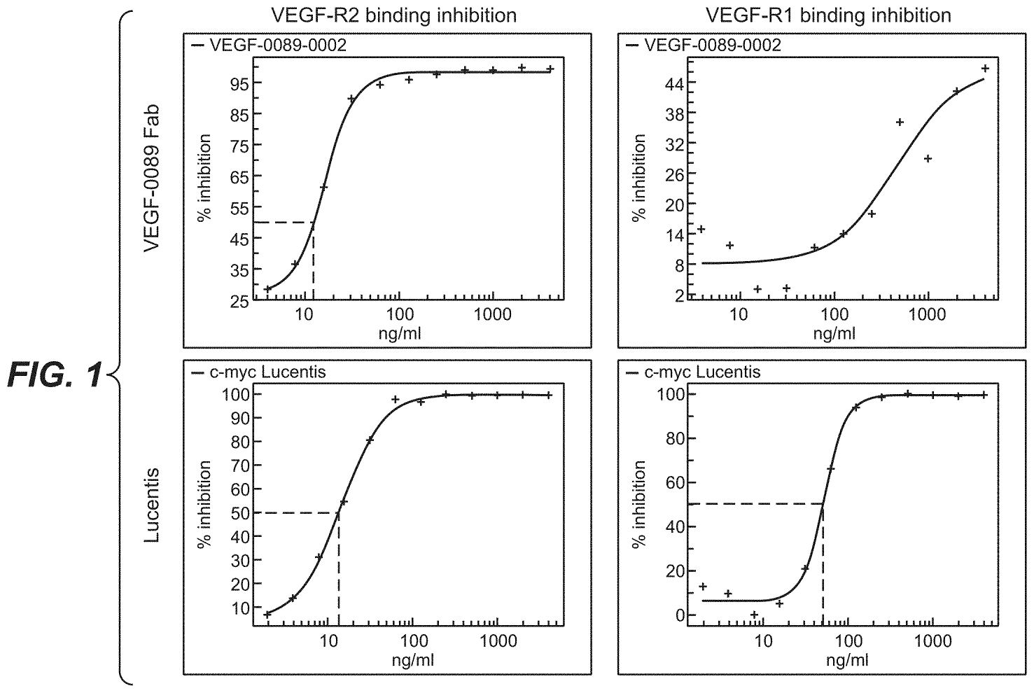

As a control and representative for a prior art anti-VEGF antibody that is used in the clinic, Lucentis® (ranibizumab, heavy chain amino acid sequence of SEQ ID NO:19, light chain amino acid sequence of SEQ ID NO:20) was assessed under the same conditions. The results are shown in FIG. 1.

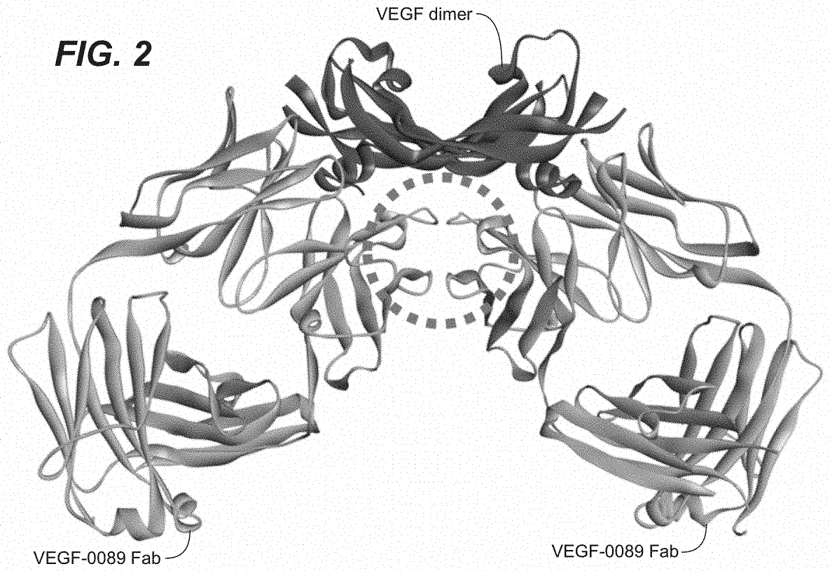

The results indicate that VEGF-0089 Fab fragment is capable of fully blocking VEGF-binding to VEGF-R2. VEGF-0089 Fab fragment did not fully block VEGF-binding to VEGF-R1. As illustrated in FIG. 2, prior art antibody Lucentis® is capable of fully blocking VEGF-binding to both receptors, VEGF-R2 and VEGF-R1.

### Example 2

X-Ray Crystallography of Antibody VEGF-0089 in Complex with VEGF-Dimer and Epitope Determination

The crystal structure of VEGF-0089 Fab fragment as described above was analyzed according to standard methods known in the art.

X-ray crystallography of VEGF-0089 Fab fragment in complex with VEGF-A121 was performed as follows:

Complex formation and purification of the dimeric complex VEGF-A121-VEGF-0089 Fab. For complex formation the VEGF-0089 Fab fragment and human VEGF-A121 (Peprotech) were mixed in a 1.1:1 molar ratio. After incubation for 16 hours overnight at 4° C. the complex was purified via gelfiltration chromatography on a Superdex200 (16/600) column in 20 mM MES, 150 mM NaCl, pH6.5. Fractions containing the dimeric complex were pooled and concentrated to 1.44 mg/ml.

Crystallization of dimeric VEGF-A121-VEGF-0089 Fab complex. Initial crystallization trials were performed in sitting drop vapor diffusion setups at 21° C. at a protein concentration of 11.5 mg/ml. Crystals appeared within 1 day out of 0.1 M Tris pH 8.5, 0.2 M LiSO₄, 1.26 M (NH4)₂SO₄. Plate shaped crystals grew in a week to a final size of 150×100×30 μm. The crystals were directly harvested from the screening plate without any further optimization steps.

Data collection and structure determination. For data collection crystals were flash cooled at 100K in precipitant solution with addition of 15% ethylene glycol as cryoprotectant. Diffraction data were collected at a wavelength of 1.0000 Å using a PILATUS 6M detector at the beamline X10SA of the Swiss Light Source (Villigen, Switzerland). Data have been processed with XDS (Kabsch, W. Acta Cryst. D66, 133-144 (2010)) and scaled with SADABS (BRUKER). The crystals belong to the space group C2 with cell axes of a=227.61 Å, b=66.97 Å, c=218.31 Å, β=104.54° and diffract to a resolution of 2.17 Å. The structure was determined by molecular replacement with PHASER (McCoy, A. J, Grosse-Kunstleve, R. W., Adams, P. D., Storoni, L. C., and Read, R. J. J. Appl. Cryst. 40, 658-674 (2007)) using the coordinates of a related in house structure of a Fab fragment and VEGF as search models. Programs from the CCP4 suite (Collaborative Computational Project, Number 4 Acta Cryst. D50, 760-763 (1994)) and Buster (Bricogne, G., Blanc, E., Brandi, M., Flensburg, C., Keller, P., Paciorek, W., Roversi, P., Sharff, A., Smart, O. S., Vonrhein, C., Womack, T. O. (2011). Buster version 2.9.5 Cambridge, United Kingdom: Global Phasing Ltd) have been used to subsequently refine the data. Manual rebuilding of protein using difference electron density was done with COOT (Emsley, P., Lohkamp, B., Scott, W. G. and Cowtan, K. Acta Cryst D66, 486-501 (2010)). Data collection and refinement statistics for both structures are summarized in table 10. All graphical presentations were prepared with PYMOL (DeLano Scientific, Palo Alto, Calif., 2002).

TABLE 1
Data collection and structure refinement statistics
Data Collection

| Wavelength (Å) | 1.0 | |
|---|---|---|
| Resolution¹ (Å) | 49.49-2.17 | (2.27-2.17) |
| Space group | C2 | |
| Unit cell (Å, °) | 227.61 66.97 218.31, 90.00 104.54 90.00 | |
| Unique reflections | 168745 | (21164) |
| Multiplicity | 3.45 | (3.43) |
| Completeness (%) | 99.8 | (99.6) |
| Mean I/σ(I) | 8.36 | (0.71) |
| R-meas | 0.073 | (0.86) |
| CC1/2 | 0.999 | (0.364) |

Refinement

| | | |
|---|---|---|
| Resolution¹ (Å) | 49.49-2.17 | (2.23-2.17) |
| Reflections used in refinement | 168674 | (12361) |
| Reflections used for R-free | 8487 | (617) |
| R-work ³ | 0.185 | (0.262) |
| R-free ⁴ | 0.227 | (0.287) |

| | |
|---|---|
| Number of atoms | 16966 |
| Protein residues | 1466 |
| RMS bonds (Å) | 0.010 |
| RMS angles (°) | 1.20 |
| Ramachandran favored (%) | 97.85 |
| Ramachandran outliers (%) | 0.15 |
| Rotamer outliers (%) | 3.47 |
| Clashscore | 2.39 |
| Average B-factor (Å²) | 65.89 |
| protein | 66.85 |
| solvent | 64.05 |

¹Values in parentheses refer to the highest resolution bins.
²R_{merge} = Σ|I − <I>|/ΣI where I is intensity.
³ R_{work} = Σ|Fₒ − <F_{c}>|/ΣFₒ where Fₒ is the observed and F_{c} is the calculated structure factor amplitude.
⁴ R_{free} was calculated based on 5% of the total data omitted during refinement.

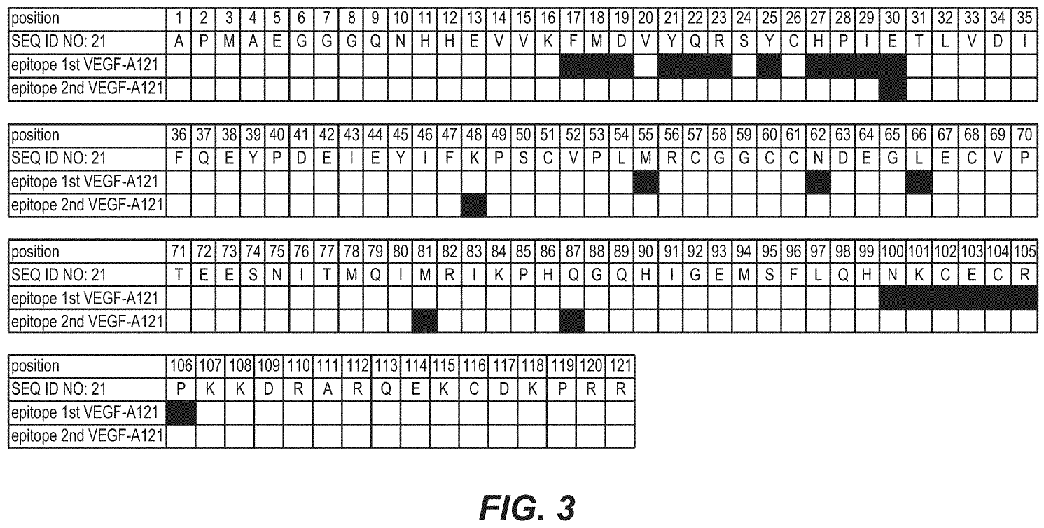

A schematic illustration of the crystal structure of two VEGF-0089 Fab fragments in complex with a human VEGF-A121 dimer is shown in FIG. 2. Amino acid residues in the VEGF-A121 dimer in contact within a distance of 5 Å with antibody VEGF-0089 Fab fragment form the conformational epitope bound by VEGF-0089 Fab on the VEGF-A121 dimer. The amino acid sequence of VEGF-A121 is SEQ ID NO: 21. An illustration of the amino acids comprised in the epitope on both VEGF-A121 molecules in the VEGF dimer is highlighted in FIG. 3.

Antibody VEGF-0089 Fab binds to the following epitope on the VEGF-A121 dimer:
in one of the individual VEGF-A121 molecules within the VEGF dimer amino acids F17, M18, D19, Y21, Q22, R23, Y25, H27, P28, I29, E30, M55, N62, L66, N100, K101, C102, E103, C104, R105 and P106; and
in the other one of the individual VEGF-A121 molecules within the VEGF dimer amino acids E30, K48, M81 and Q87.

An overlay of the crystal structures of a human VEGF-dimer in complex with VEGF-R1 domain 2 and in complex with VEGF-0089 Fab fragment is depicted in FIG. 4. This superimposition illustrates that the light chain of the VEGF-0089 antibody of the invention superimposes with domain 2 of VEGF-R1. An overlay of the crystal structures of a human VEGF-dimer in complex with VEGF-R2 domains 2 and 3, and in complex with VEGF-0089 Fab fragment is depicted in FIG. 5. This superimposition illustrates that the light chain of the VEGF-0089 antibody of the invention superimposes with domain 2 of VEGF-R2.

Consequently, the x-ray data show that the epitope bound by the VEGF-0089 antibody overlaps with the region of VEGF that binds to VEGF-R1 and VEGF-R2.

From the tertiary structure of the VEGF-dimer-antibody-complex shown in FIG. 2 it can be seen that two regions within the antibody's VH domain, i.e. H-CDR2 and H-FR3, are in close spatial proximity to each other.

This might sterically hinder the binding of a second antibody to the VEGF-dimer in case one Fab fragment is already bound. Without being bound to this theory, the observation that the antibody is not capable of (fully) blocking VEGF-binding to VEGF-R1 may result from this suspected steric hindrance. VEGF-dimers, to which only one Fab fragment rather than two have been bound are expected to still be able to bind to VEGF-R1, as VEGF-R1 is known to be bound by VEGF with strong affinity. Of note, VEGF-R2 is known to be bound by VEGF with weak affinity, supporting the observation that VEGF-binding to VEGF-R2 is blocked by VEGF-0089.

### Example 3

### Provision of Variants of VEGF-0089

Based on the theory explained above, the VEGF-0089 antibody was modified in order to facilitate simultaneous binding of two antibody molecules to the VEGF-dimer.

Based on this theory, the following antibody variants of antibody VEGF-0089 were generated.

TABLE 1
Amino acid sequences and amino acid modifications
in generated candidate and control antibodies

| | | | SEQ ID NO: | |
|---|---|---|---|---|
| | H-CDR2 | H-FR3 | VH | VL |
| VEGF-0089 | wt | wt | 1 | 2 |
| Candidate antibodies | | | | |
| VEGF-0112 | N52aS | wt | 12 | 2 |
| VEGF-P1AD8674 | wt | D72N, S74A, | 15 | 2 |
| | | K75E | | |

Fab fragments of the antibodies were cloned and expressed as described in Example 1. Amino acid sequences of heavy chains and light chains are shown in Table 2.

TABLE 2
Amino acid sequences of anti-VEGF antibodies

| | heavy chain | light chain |
|---|---|---|
| VEGF-0089 Fab | SEQ ID NO: 10 | SEQ ID NO: 11 |
| VEGF-0112 Fab | SEQ ID NO: 14 | SEQ ID NO: 11 |
| VEGF-P1AD8674 Fab | SEQ ID NO: 17 | SEQ ID NO: 11 |

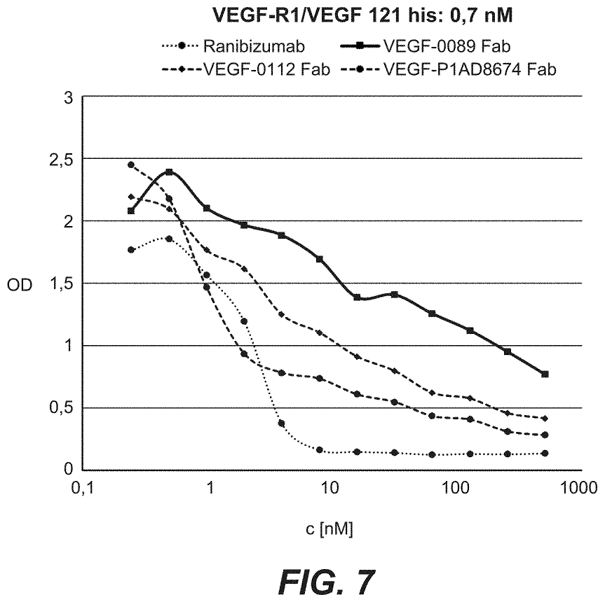

Binding of VEGF to VEGF-R1 as well as VEGF-R2 in presence of improved antibody Fab fragments was tested as described in Example 1. All antibody Fab fragments listed in Table 2 were tested. Results are shown in FIGS. 6 and 7.

### Example 4

### VEGF-Binding Affinity of Improved Variants of VEGF-0089

The affinity of the antibodies was determined by SPR using the same methods as described in Example 1. Results for indicated antibodies are shown in Table 3.

TABLE 3
Affinities of anti-VEGF antibodies

| | KD [pM] at 25° C. | KD [pM] at 37° C. |
|---|---|---|
| VEGF-0089 Fab | 143 | 110 |
| VEGF-0112 Fab | 74 | 85 |

1. A method of improving inhibition of VEGF binding to VEGF-R1 of an antibody that binds to VEGF comprising an antigen binding site formed by cognate pair of a VH and a VL domain, wherein the antibody binds to an epitope of VEGF that overlaps with the VEGF-R1-binding region and the VEGF-R2-binding region in the VEGF molecule, the method comprising the steps of
a) providing an analysis of the tertiary structure of a complex of a VEGF-dimer bound by a first and a second antigen binding site of said antibody that binds to VEGF (VEGF-dimer-antibody-complex);
b) identifying at least one amino acid residue located in the VH domain or VL domain of said antibody, wherein said amino acid residue within the first antigen binding site and said amino acid residue within the second antigen binding site are spatially arranged in close proximity in the VEGF-dimer-antigen-complex; and
c) substituting said at least one amino acid residue identified in step b) by
i) an amino acid having a smaller side chain volume; and/or
ii) an amino acid having a side chain of different charge.

2. The method of claim 1, wherein the antibody binds to the same or overlapping epitope than an antibody characterized by a VH of SEQ ID NO:01 and a VL of SEQ ID NO:02.

3. The method of one of the preceding claims, wherein the antibody binds to the same or an overlapping epitope to a conformational epitope on a dimer of VEGF-A121, wherein VEGF-A121 comprises an amino acid sequence of SEQ ID NO: 21, wherein the epitope comprises
in one of the individual VEGF-A121 molecules within the VEGF dimer amino acids F17, M18, D19, Y21, Q22, R23, Y25, H27, P28, I29, E30, M55, N62, L66, N100, K101, C102, E103, C104, R105 and P106; and
in the other one of the individual VEGF-A121 molecules within the VEGF dimer amino acids E30, K48, M81 and Q87.

4. The method of one of the preceding claims, wherein the amino acid with a smaller side chain volume is selected from D, E, S, T, N, G, A, V, I, and L.

5. The method of one of the preceding claims, wherein an amino acid with a side chain of positive charge is substituted by an amino acid having a side chain of negative charge or by an amino acid having an uncharged side chain.

6. The method of one of the preceding claims, wherein an amino acid with a side chain of negative charge is substituted by an amino acid having a side chain of positive charge or by an amino acid having an uncharged side chain.

7. The method of one of the preceding claims, wherein the at least one substituted amino acid residue is located in the heavy chain variable domain of said antibody.

8. The method of one of the preceding claims, wherein the at least one substituted amino acid residue is located within a heavy chain CDR of said antibody.

9. The method of one of the preceding claims, wherein the at least one substituted amino acid residue is located within H-CDR2 of said antibody.

10. The method of one of the preceding claims, wherein the at least one amino acid residue replaced is located within a heavy chain FR of said antibody.

11. The method of one of the preceding claims, wherein the at least one amino acid residue replaced is located within H-FR3 of said antibody.

12. The method of one of the preceding claims, wherein the antibody comprises comprises (a) CDR-H1 comprising the amino acid sequence of SEQ ID NO:03; (b) CDR-H2 comprising the amino acid sequence of SEQ ID NO:04; (c) CDR-H3 comprising the amino acid sequence of SEQ ID NO:05; (d) CDR-L1 comprising the amino acid sequence of SEQ ID NO:06; (e) CDR-L2 comprising the amino acid sequence of SEQ ID NO:07; and (f) CDR-L3 comprising the amino acid sequence of SEQ ID NO:08.

13. The method of one of the preceding claims, wherein the antibody comprises a VH comprising SEQ ID NO:01 and a VL comprising SEQ ID NO:02.

14. An antibody provided by a method of one of the preceding claims.

15. An antibody that binds to VEGF having a VH domain comprising SEQ ID NO:12 and a VL domain comprising SEQ ID NO: 2.

16. An antibody that binds to VEGF having a VH domain comprising SEQ ID NO:15 and a VL domain comprising SEQ ID NO: 2.
